(19) United States
(12) Patent Application Publication
(10) Pub. No.: US 2022/0270714 A1
(43) Pub. Date: Aug. 25, 2022

(54) MOLECULAR STRUCTURE GENERATION METHOD AND NON-TRANSITORY COMPUTER-READABLE MEDIUM STORING PROGRAM

(71) Applicant: HPC SYSTEMS Inc., Tokyo (JP)

(72) Inventors: Takuya Okamoto, Kyoto-shi (JP); Yukihiro ABE, Kyoto-shi (JP); Seiji UENO, Kyoto-shi (JP)

(21) Appl. No.: 17/650,684

(22) Filed: Feb. 11, 2022

(30) Foreign Application Priority Data
Feb. 12, 2021 (JP) 2021-020762

(51) Int. Cl. G16C 20/70; G16C 20/50

(52) U.S. Cl. CPC G16C 20/70; G16C 20/50

(57) ABSTRACT

To provide a molecular structure generation method and a non-transitory computer-readable medium storing a program capable of generating various molecular structures while satisfying desired property values so as not to be localized around a specific molecular structure.

A molecular structure generation method according to the present invention includes: a selection step of classifying a plurality of initial molecules prepared in advance into clusters based on a feature amount and selecting a starting molecule having a maximum confidence limit value from each of the classified clusters. The method further includes an evolutionary development step of evolving each of the starting molecules. Further the selection step and the evolutionary development step are repeatedly executed for all molecules including the initial molecules and the evolved starting molecules to generate a new molecular structure.

### INCORPORATION BY REFERENCE

This application is based upon and claims the benefit of priority from Japanese patent application No. 2021-20762, filed on Feb. 12, 2021, the disclosure of which is incorporated herein in its entirety by reference.

### BACKGROUND

The present invention relates to a molecular structure generation method and a non-transitory computer-readable medium storing a program.

The development of conventional functional materials is performed based on a direct problem. Specifically, researchers and developers imagine molecular structures considered to have desired properties, estimate the properties of the molecular structures by simulation according to the molecular orbital (MO) method or the molecular dynamics (MD) method and an empirical method such as the atomic group contribution method based on databases, and find suitable molecular structures by screening. Furthermore, methods of estimating properties in a short time using machine learning (ML) based on a large amount of data without relying on the MO method or MD method have been developed and started to be used at the research and development site of functional materials. The molecular structure to be generated depends on the experience, intuition and insight of the researchers and developers.

On the other hand, inverse problem research and development to estimate and develop a molecular structure having desired properties without relying on the intuition and experience has begun to become active. As a method using deep learning (DL), there is a method of learning by stacking a plurality of layers of neural networks (NN) on a database and using it for model creation. A convolutional neural network (CNN) is also used to handle molecular structures and the like. Further, a recurrent neural network (RNN) is used for handling character string data expressing an organic compound. Further, as for graph data, a graph neural network (GNN) and a graph convolutional neural network (GCN) have begun to be effectively applied.

Non-Patent Document 1 discloses a method involving a direct problem to create a prediction model that associates molecular structures and their properties using data made up of a huge number of molecular structures and properties to predict the properties of a given molecular structure and an inverse problem to derive a molecular structure satisfying desired properties.

Examples of the method involving the reverse problem to derive a molecular structure satisfying desired properties include a genetic algorithm (GA), a Monte Carlo tree search method (MCTS), and the like. A molecular structure is represented by a character string by the simplified molecular input line entry system (SMILES) method.

The first important issue of the inverse problem is how to generate a structure that realizes a desired property value. A molecular structure to be actually synthesized is virtually created, and the property value is predicted based on a regression model created by machine learning or the like. As one of the approach methods, Non-Patent Documents 1 to 4 disclose a method of expressing a regression model under a constraint condition x by a probability f(y|x), estimating the variables having a posterior distribution f(x|y) by the Bayesian theorem, and extracting a structure satisfying the variables.
[Non-Patent Document 1] H. Ikebata, K. Hongo, T. Isomura, R. Maezono, and R. Yoshida, J. Comput. Aided Mol. Des., 31, 379 (2017).
[Non-Patent Document 2] T. Miyao, M. Arakawa, and K. Funatsu, Molecular Informatics, 29, 111 (2010).
[Non-Patent Document 3] T. Miyao, H. Kaneko, and K. Funatsu, Molecular Informatics, 33, 764 (2014).
[Non-Patent Document 4] X. Yang, Z. Zhang, K. Yoshizoe, K. Terayama, and K. Tsuda, Sci. Technol. Adv. Mater. 18, 972 (2017).
[Non-Patent Document 5] X. Q. Lewell, D. B. Judd, S. P. Watson, and M. M. Hann, J. Chem. Inf. Comput. Sci. 1998, 38, 3, 511-522
[Non-Patent Document 6] J. Degen, C. Wegscheid-Gerlach, and M. Rarey, ChemMedChem, 3 (10), 1503 (2008).
[Non-Patent Document 7] K. Kim, S. Kang, J. Yoo, Y. Kwon, Y. Nam, D. Lee, I. Kim, Y. Choi, Y. Jung, S. Kim, W. Son, J. Son, H S Lee, S. Kim, J. Shin, and S. Hwang, npj Computational Materials, 4, 67 (2018).

### SUMMARY

The important thing required for generating a virtual structure under constraint conditions is to generate various structures including new structures that have not been developed so far. Using the molecular structure generation methods developed so far, there is a tendency that once a structure satisfying desired property values is found, a large number of similar molecular structures around it are generated. In this case, even if the required properties are satisfied, it is necessary to give up using this molecular structure because the synthesis method is difficult, the raw material is difficult to obtain, it cannot be manufactured by the existing production facilities, or it is expensive. Thus, it is necessary to generate another molecular structure again using some method.

An object of the present invention is to provide a molecular structure generation method and a non-transitory computer-readable medium storing a program capable of generating various molecular structures while satisfying desired property values so as not to be localized around a specific molecular structure.

An aspect of the present invention provides a molecular structure generation method including: a selection step of classifying a plurality of initial molecules prepared in advance into clusters based on a feature amount and selecting a starting molecule having a maximum confidence limit value from each of the classified clusters; and an evolutionary development step of evolving each of the starting molecules, wherein the selection step and the evolutionary development step are repeatedly executed for all molecules including the initial molecules and the evolved starting molecules to generate a new molecular structure.

Another aspect of the present invention provides a molecular structure generation method including: a selection step of selecting a starting molecule having a maximum confidence limit value from a plurality of initial molecules prepared in advance; and an evolutionary development step of evolving each of the starting molecules, wherein the selection step and the evolutionary development step are repeatedly executed for all molecules including the initial molecules and the evolved starting molecules to generate a new molecular structure.

Another aspect of the present invention provides a molecular structure generation method including: a selection step of calculating a feature amount of each of a plurality of initial molecules prepared in advance and further selecting a starting molecule according to a probability value calculated based on the feature amount; and an evolutionary development step of evolving each of the starting molecules, wherein the selection step and the evolutionary development step are repeatedly executed for all molecules including the initial molecules and the evolved starting molecules to generate a new molecular structure.

According to the present invention, it is possible to provide a molecular structure generation method and a non-transitory computer-readable medium storing a program capable of generating various molecular structures while satisfying desired property values so as not to be localized around a specific molecular structure.

The above and other objects, features and advantages of the present disclosure will become more fully understood from the detailed description given below and the accompanying drawings which are given by way of illustration only, and thus are not to be considered as limiting the present disclosure.

### BRIEF DESCRIPTION OF DRAWINGS

FIG. 1 is a schematic diagram of a molecular structure generation method according to the present invention.

FIG. 2 is a diagram showing the relationship between a graph structure, a molecular structure, and a phylogenetic tree in the present invention.

FIG. 3 is a diagram showing definitions of first and second desired regions of property values in the present invention.

FIG. 4 is a diagram showing a flow of a molecular clustering process in the present invention.

FIG. 5 is a conceptual diagram showing a molecular structure generation method according to a first embodiment of the present invention.

FIG. 6 is a diagram showing a flow of a process of generating a molecular structure according to the first embodiment of the present invention.

FIG. 7 is a diagram showing the results of principal component analysis for the molecular structure generated using the molecular structure generation method according to the first embodiment of the present invention.

FIG. 8 is a conceptual diagram showing a molecular structure generation method according to a second embodiment of the present invention.

FIG. 9 is a diagram showing a flow of a process of generating a molecular structure according to the second embodiment of the present invention.

FIG. 10 is a diagram showing the results of principal component analysis for the molecular structure generated using the molecular structure generation method according to the second embodiment of the present invention.

FIG. 11 is a conceptual diagram showing a molecular structure generation method according to a third embodiment of the present invention.

FIG. 12 is a diagram showing a flow of a process of generating a molecular structure according to the third embodiment of the present invention.

FIG. 13 is a diagram showing the results of principal component analysis for a molecular structure generated using the molecular structure generation method according to the third embodiment of the present invention.

FIG. 14 is a conceptual diagram showing a method for generating a molecular structure using a genetic algorithm method according to a conventional example.

FIG. 15 is a diagram showing the results of principal component analysis for a molecular structure generated using a genetic algorithm method according to a conventional example.

FIG. 16 is a block diagram showing a hardware configuration example for realizing the process related to the molecular structure generation method according to the present invention.

### DESCRIPTION OF EMBODIMENTS

Hereinafter, embodiments will be described with reference to the drawings. Since the drawings are simplified, the technical scope of the embodiment should not be narrowly interpreted based on the description of the drawings. The same elements are designated by the same reference numerals, and duplicate description will be omitted.

### <Molecular Structure Generation Method According to Embodiment>

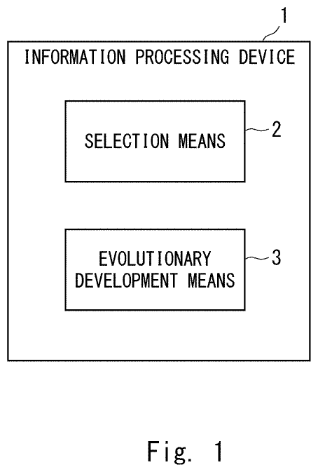

A molecular structure generation method according to an embodiment will be described with reference to FIGS. 1 to 4. FIG. 1 is a schematic diagram of a molecular structure generation method according to an embodiment.

The molecular structure generation method according to the embodiment includes a selection means 1 for classifying a plurality of initial molecules prepared in advance into clusters based on a feature amount and selecting starting molecules having the maximum confidence limit value from the classified clusters and an evolutionary development means 2 for evolving each of the starting molecules.

The selection means 1 may select a starting molecule having the maximum confidence limit value from a plurality of initial molecules prepared in advance. The selection means 1 may calculate a feature amount of each of the plurality of initial molecules prepared in advance, and further select a starting molecule according to a probability value calculated based on the feature amount.

In the molecular structure generation method according to the embodiment, a new molecular structure is generated by repeatedly executing the selection means 1 and the evolutionary development means 2 for all the molecules including the initial molecules and the evolved starting molecules. The selection means 1 and the evolutionary development means 2 may be processed by an information processing device 1 or may be executed in a system using a plurality of devices.

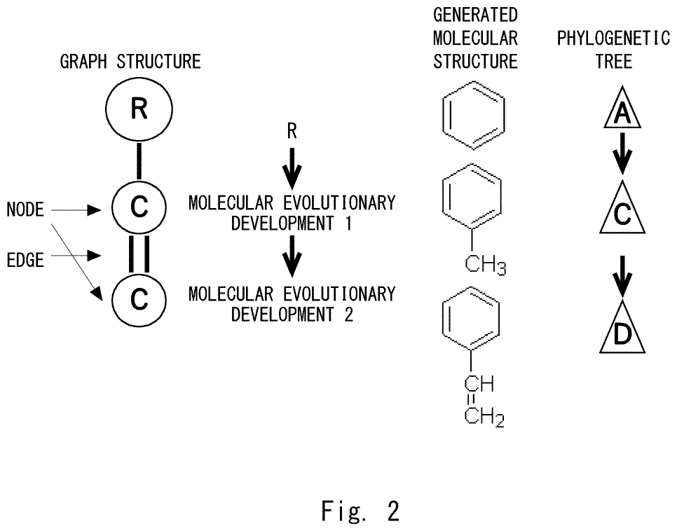

FIG. 2 is a diagram showing the relationship between a graph structure, a molecular structure, and a phylogenetic tree in the embodiment. As shown in FIG. 2, the molecular structure is shown using a graph notation in which the atoms constituting a molecule are represented as nodes and the bonds between the atoms are represented as edges. The starting molecular structure R is, for example, benzene, and is registered as the starting molecule A in the phylogenetic tree. When a carbon atom is added in molecular evolutionary development 1, toluene is generated and added to the phylogenetic tree as a molecule C. When a carbon atom is further added via a double bond, styrene is generated in molecular evolutionary development 2, and is added to the phylogenetic tree as a molecule D. At this time, single bonds and double bonds are handled as edges. The molecular evolutionary development means the generation of a new molecule by adding an atom to an original molecule.

As the dataset in which molecular structures are recorded, for example, publicly available PubChem, PubChemQC, ZINC, ChemSpider, Chembl, GDB, QM7, QM8, QM9 and the like can be used, but the dataset is not limited thereto.

The performance of a molecule with respect to desired properties is evaluated using a score. The score is a numerical value indicating how much desired properties are satisfied, and is calculated as an acquisition function. The molecular structure having the maximum acquisition function is selected as the next compound to be evolved.

a1 molecular structures stored in a data frame are classified into f1 types of clusters CL(1) to CL(f1) according to the feature amounts of the molecular structures calculated for each molecule. The details of the calculation of the feature amount of the molecular structure will be described later. a1 is an integer of 1 or more, preferably in the range of 30 to 1,000,000,000, and more preferably in the range of 100 to 1,000,000,000. f1 is an integer of 2 or more, preferably in the range of 3 to 10,000, and more preferably in the range of 5 to 10,000.

The molecular score may be calculated using a confidence limit UCB1ᵢ value expressed using the following equation (1) or MScᵢ expressed using the equation (2). The MScᵢ represented by the equation (2) is used in a third embodiment described later. Scores are compared in the same cluster classified into f1 types, and the molecule having the maximum score is selected as the starting molecule.

In the third embodiment to be described later, evolutionary development may be caused by crossover-reaction or mutation by adding an arbitrary atom to the selected starting molecule, replacing an atom at an arbitrary position with another atomic species, and adding a fragment generated by fragmentation of a molecule selected from the molecules other than the starting molecule to generate a new molecule, which may be added to the phylogenetic tree of the starting molecule. At this time, the fragmented molecule is selected based on the probability calculated using the equation (3) or (4) that probabilistically expresses the score of the molecule among the molecules other than the starting molecule of interest.

As the fragmented molecule, b1 molecules are selected from a1 molecules by the probability Prᵢ calculated using the equation (3) or (4).

$$\begin{matrix}[ Math . 1 ] & \\ UCB {1}_{i} = {\overline{x}}_{i} + C \sqrt{\frac{ln ( n )}{{n}_{i}}} & ( 1 )\end{matrix}$$

Here, the logarithm part may be a common logarithm. C is an arbitrary real number. Further, n is the sum of the number of molecules initially read and the number of molecules generated, and nᵢ is the number of all molecules generated after the molecule to be calculated and added to the same phylogenetic tree. The average value of xᵢ in the equation (1) represents the average value of the scores of all the molecules generated after the molecule to be calculated and added to the same phylogenetic tree.

[Math. 2]

MScᵢ=(1−λ)g(Scᵢ)+Δh(n₂ᵢ) (2)

Here, Scᵢ represents the score of the molecule i, and λ represents the weight, which is an arbitrary real number of 0.0 to 1.0. Further, g and h represent Gaussian functions. n₂ᵢ represents the number of adjacent molecules in the phylogenetic tree to which the molecule for which the score is to be calculated belongs.

$$\begin{matrix}[ Math . 3 ] & \\ {Pr}_{i} = \frac{UCB {1}_{i}}{{\sum }_{i = 2}^{n} {e}^{{S}_{{c}_{i}}}} & ( 3 ) \\ [ Math . 4 ] & \\ {Pr}_{i} = \frac{{MS}_{{c}_{i}}}{{\sum }_{i = 1}^{n} {MS}_{{c}_{i}}} & ( 4 )\end{matrix}$$

Here, n represents the number of molecules to be compared.

The score of a molecule is expressed as a score Sc in which in the simplest form of an acquisition function, the molecular structure of interest simply satisfies desired properties. Sc may be obtained for a single property, or may be the sum of scores for a plurality of properties that are desired to be satisfied at the same time.

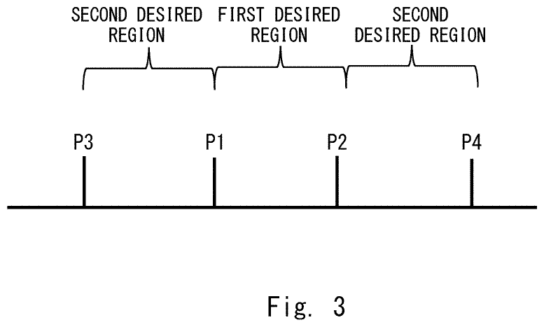

Here, the first desired region and the second desired region regarding the property values will be described with reference to FIG. 3. FIG. 3 is a diagram showing the definitions of the first and second desired regions of the property values in the embodiment.

In FIG. 3, P1 to P4 are the property values of a molecule. In FIG. 3, the first desired region is P1 to P2. The first desired region is a desired property region. Further, a wide range P3 to P4 including the P1 to P2 region, which is the first desired region, is defined as the second desired region. If the property value a estimated by a method such as a model representing the relationship between the molecule i and the properties of a certain molecular structure, a molecular orbital method, or a molecular dynamics method are in P1 to P2, the score Si is set to 1.0. If the property value a is in P3 to P1 or P2 to P4, the Si is calculated using the equation (5). When the property value a includes a plurality of property values, the score Si corresponding to the property value ai is added with a weight wi, and is calculated by the equation (6) so that the total value becomes 1.0. Here, i is an integer of 1 or more, and n is the number of property values to be satisfied at the same time.

$$\begin{matrix}[ Math . 5 ] & \\ {S}_{i} = \frac{\vert P 3 - a \vert}{\vert P 1 - P 3 \vert} or {S}_{i} = \frac{\vert a - P 2 \vert}{\vert P 3 - P 2 \vert} & ( 5 ) \\ [ Math . 6 ] & \\ {Sc}_{i} = {\sum }_{i = 1}^{n} {S}_{i} {w}_{i} & ( 6 )\end{matrix}$$

In addition to the score based on the above-mentioned properties, a synthetic accessibility (SA) score may be used as a score based on the synthesizability of the molecule. The SA score is a real number evaluated from 1 to 10 based on the appearance frequency of the ECFP4 fingerprints of 1-million molecular structures in PubChem, and the closer it is to 1, the easier it is to synthesize the molecule.

The improvement probability PI calculated using the equation (7) may be used as the acquisition function. When it is desired to maximize the property value, the improvement probability PI is calculated by the integral value of the probability density function in the portion of the predicted probability distribution obtained for the sample, which is higher than the known maximum value yₘₐₓ of the property value.

[Math. 7]

PI(x*)=∫_{y}_{ₘₐₓ}^{∞}N(f|μx^{*2}),σ(x*))df (7)

Here, x* is the optimum solution, f is a random variable, and f˜N(f|μ, σ²) are the prediction results by the Gaussian process. The random variable f follows a normal distribution having an average value μ and a variance σ².

The acquisition function may be expressed using the expected improvement degree EI shown in the following equation (8).

$$\begin{matrix}[ Math . 8 ] & \\ EI ( {x}^{*} ) = \{ \begin{matrix}( μ ( {x}^{*} ) - {y}_{max} ) Φ ( Z ) + σ ( {x}^{*} ) ϕ ( Z ) & if σ ( {x}^{*} ) > 0 \\ 0 & if σ ( {x}^{*} ) = 0\end{matrix} & ( 8 )\end{matrix}$$

Here, Φ(Z) is a cumulative density function, and returns a value obtained by integrating the probability density function within a certain range of random variables. φ(Z) represents the probability density function, and Z represents ((yₘₐₓ−μ)/σ(x*).

The acquired value may be calculated using UCB1 (UCB: Upper Confidence bound) represented by the equation (1). The probability Prᵢ, which is probabilistically expressed based on the score of the molecule, is calculated by the equation (3) or (4).

The properties of each molecule can be estimated using a model equation derived by statistical processing or machine learning from a dataset consisting of molecular structures and property values. The properties of each molecule can be calculated using a molecular orbital method, a molecular dynamics simulation, and an atomic group contribution method when the dataset is not used. The properties of each molecule may be calculated by combining some of these calculation methods.

Molecular evolutionary development is carried out by mutation of one molecule and crossover-reaction between multiple molecules. The evolutionary development is carried out by selecting any part of a starting molecule as the reaction site and adding or removing one fragment or one heavy atom, or substituting any heavy atom and changing the bonding form. Specifically, mutations refer to, for example, a change to a —COOH group due to the replacement of the N atom of a —NO₂ group with the C atom, a change to ethane due to the change of a double bond of ethylene to a single bond, and the formation of butane due to the elimination of two C atoms from cyclohexane. The crossover-reaction between multiple molecules refers to, for example, a reaction in which the C atoms at both ends of butadiene produced by the elimination of ethylene from benzene are added to the second and third positions of the naphthalene molecule to form anthracene, benzene is eliminated from biphenyl and added to the first position of naphthalene to produce 1-phenylnaphthalene, and biphenyl itself is added to the 2 position of naphthalene to produce 2-biphenylnaphthalene. Whether the evolutionary development of molecules will adopt mutations such as fragment addition, heavy atom addition, or heavy atom substitution, or crossover-reaction between multiple molecules depends on a probability predetermined each time.

Fragmentation of molecules can be performed using RECAP (Retro synthetic Combinatorial Analysis Procedure) or BRICS (Breaking of Retrosyntheticly Interesting Chemical Substructures) rules. The fragmentation of molecules may be carried out by adding a linker and a fragment extracted from an existing molecular structure to evolve the molecule. These methods are disclosed in Non-Patent Documents 5-7.

For example, when RECAP is used, an organic molecule is decomposed into fragments at positions where a bond in the molecule is easily broken, focusing on each bond of amide, ester, amine, N—C in urea, ether, C═C, ammonium, N—S in sulfanamide, aromatic ring-aromatic ring, N (inside aromatic ring)-C (sp3), and N (inside lactam ring)-C (sp3). When BRICS is used, a molecule is decomposed into fragments, focusing on 16 types of bonds by the same method as RECAP.

The existing molecule may be fragmented to any size. Specifically, for example, aniline is fragmented into an amino group and a phenyl group, and ethanol is fragmented into an ethyl group and a hydroxy group. Cyclocyclic compounds such as cyclohexane and ethylene oxide; heterocyclic compounds such as furan, thiophene, pyrrole, oxazole, thiazole; condensed ring compounds such as inden, naphthalene, fluorene, phenanthrene, anthracene, pyrene, chrysene, naphthacene, thiazole, oxazole, xanthene, aclysine, phenoxazine, dibenzofuran, indole, benzofuran, quinoline, and naphthoquinone; spiro ring compounds such as spiro[4,4]nonane and spiro[4,5]decane; atomic group such as nitro group, azo group, carbonyl group, thiocarbonyl group, and carbino group can be used as chemically meaningful fragments or linkers without being decomposed. In these fragmentations, the number of sites where each fragment can bind to the starting molecule may be any number of one or more.

The heavy atom constituting the starting molecule may be substituted with any heavy atom such as C, O, N, S, Si, B, Cl, F, Br, Cu, Fe, Zn and Mg. However, heavy atoms are not limited to these atoms.

Clustering of molecules may be performed based on molecular similarity. The molecular similarity is determined by the feature amounts of the molecules or the distance between the molecules.

As a method for calculating the feature amount of the molecular structure, for example, a fingerprint that compresses a chemical structure into several thousand fixed-length vectors and represents it by a bit string of 0 and 1 may be used. As the fingerprint, for example, MACCS Key, Topological fingerprint, Morgan fingerprint, MinHash fingerprint, Avaron fingerprint, AtomPair fingerprint, DonarAcceptor fingerprint, Extended Connectivity fingerprint, Functional Connectivity fingerprint, Dragon Fingerprint, and the like may be used. In addition, using fingerprint, descriptors such as RDkit descriptors and Mordred descriptors, a graph kernel in vector notation with an infinite number of elements to be added, the number of electrons determined for each atom by the graph itself, atomic feature amounts such as bond information, and the like can be quantified. However, the calculated feature amount of the molecular structure is not limited to these.

As a method for evaluating the similarity between molecules A and B, the Tanimoto coefficient S_{AB} is used.

$$\begin{matrix}[ Math . 9 ] & \\ {S}_{AB} = \frac{c}{a + b - c} & ( 9 )\end{matrix}$$

Here, a is the number of “1” in the bit array of A's fingerprint, b is the number of “1” in the bit array of molecule B, and c is the number of “1” common to A and B.

The intramolecular distance D_{AB} between A and B is calculated using the following equation (10).

[Math. 10]

D_{AB}=1−S_{AB} (10)

The distance between molecules may be calculated using Chebyshev Distance, Euclidean Distance, Manhattan Distance, Mahalanobis Distance, or the like. The distance d between the i-th molecule and the j-th molecule is calculated using the following equations (11) to (14) in which xₖ⁽ⁱ⁾ is set as the k-th variable in the i-th molecule.

When Euclidean Distance is used, the distance between molecules is calculated using the following equation (11).

[Math. 11]

d_{i,j}={square root over (Σₖ₌₁^{m}(xₖ⁽ⁱ⁾−xₖ^{(j)})²)} (11)

When Chebyshev Distance is used, the distance between molecules is calculated using the following equation (12).

[Math. 12]

d_{i,j}=maxₖ(|xₖ⁽ⁱ⁾−xₖ^{(j)}|) (12)

When Manhattan Distance is used, the distance between molecules is calculated using the following equation (13).

[Math. 13]

d_{i,j}=Σₖ₌₁^{m}|xₖ⁽ⁱ⁾−xₖ^{(j)}| (13)

When Maharanobis Distance is used, the distance between molecules is calculated using the following equation (14).

[Math. 14]

d_{i,j}=\sqrt{(x⁽ⁱ⁾−mₓ)Σ⁻¹(x⁽ⁱ⁾−mₓ)^{T}} (14)

Here, x⁽ⁱ⁾ and x⁽ⁱ⁾ are vectors in which the values of the variables of the i-th and j-th molecules are stored, mₓ is a vector in which the average value of the variables is stored, and Σ⁻¹ represents a variance-covariance matrix.

As a clustering method, for example, a k-Means method, a k-Means++ method, or a Gaussian Mixture method is used. The k-means method is a method for classifying molecules into k clusters, and is calculated as follows.

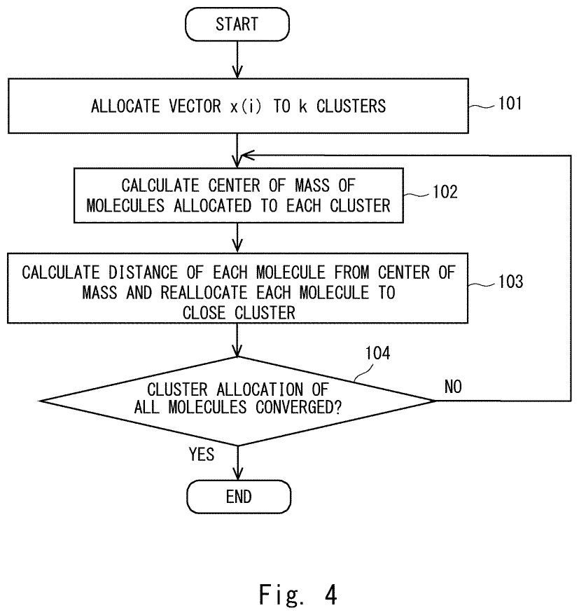

Here, the method of clustering molecules will be described with reference to FIG. 4. FIG. 4 is a diagram showing a flow of a molecular clustering process, and is for example, a flow when the k-means method is used. First, the vector x⁽ⁱ⁾ is randomly allocated to k clusters (step 101). Next, the center of mass is calculated for the molecule allocated to each cluster (step 102). Further, for each molecule, the distance from the center of mass calculated in step 102 is calculated, and the vector x(i) is reallocated to the cluster having the closest distance (step 103). The processes of steps 102 and 103 are repeated until the allocation of clusters of all molecules converges (YES in step 104).

Assuming that the set of indices of the molecules belonging to the j-th cluster is I, the center of mass Gⱼ of the j-th cluster is calculated by the following equation (15).

$$\begin{matrix}[ Math . 15 ] & \\ {G}_{j} = \frac{1}{\vert {I}_{j} \vert} {\sum }_{i}^{n} {x}^{( i )} & ( 15 )\end{matrix}$$

As a method of visualizing the molecular structure generated by clustering, for example, principal component analysis (PCA) can be mentioned. When PCA is used, since given data is projected onto a lower-dimensional space by performing rotational transform of a coordinate system around a sample average, the data can be visualized so that scattering of points is seen as large as possible with fewer coordinate axes.

As a method for non-linear dimensional reduction of high-dimensional data to two or three dimensions, for example, the t-SNE (t-distributed stochastic neighbor embedding) method for maintaining the distance relationship between molecules and GTM (generative topographic mapping) for maintaining the positional relationship between molecules are used.

### First Embodiment

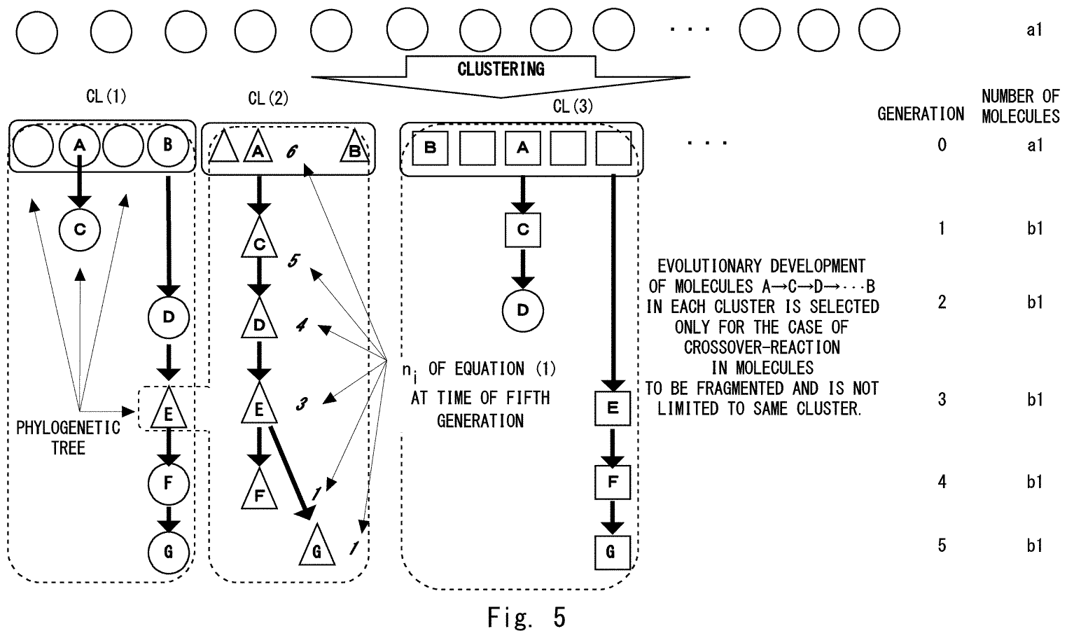

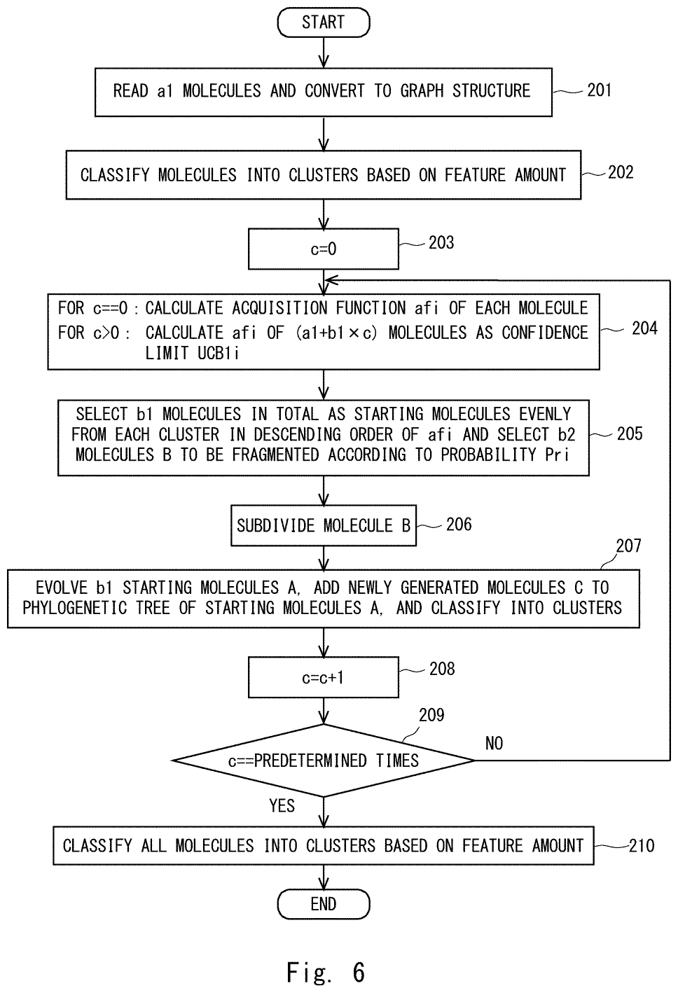

The molecular structure generation method according to the present embodiment will be described with reference to FIGS. 5 and 6. FIG. 5 is a conceptual diagram showing a molecular structure generation method according to the present embodiment. FIG. 6 is a flowchart of a process of generating a molecular structure in the present embodiment. In the present embodiment, the desired property value is PR.

In the molecular structure generation method of the present embodiment, as shown in FIG. 5, first, any a1 molecules are clustered. a1 may be, for example, 1,000, but is not limited to this. Clustering is to characterize a1 molecules by its structure and classify the molecules. The classified clusters include f1 types of CL(1) to CL(f1), and the cluster classification is the 0th generation. In each cluster, each molecule is evolved to generate b1 molecules. The evolutionary development of each molecule may be carried out by selecting one molecule having the largest UCB1ᵢ evenly from each cluster. By repeating these processes a plurality of times, a predetermined number of molecules are generated.

The flow of the process of generating the molecular structure in the present embodiment will be described with reference to FIG. 6. First, a1 molecular structures are read from a database in which molecular structures are stored, and converted to a graph structure that expresses a molecular structure using the atoms constituting the molecule as nodes and the bonds between atoms as edges and stored in a data frame (step 201). The a1 molecular structures stored in the data frame are classified into f1 types of clusters CL(1) to CL(f1) according to the feature amount calculated using, for example, fingerprint, for each molecule (step 202). The cluster classification corresponds to the 0th generation.

The acquisition function afᵢ is calculated for each of the a1 molecules using the equation (16) (step 204).

[Math. 16]

afᵢ=sᵢ+c\sqrt{ln(a1)} (16)

Here, sᵢ is the score of the i-th molecule calculated using the equations (5) and (6), and c is a constant, and for example, √2 or the like is used.

b1 molecules are selected as the starting molecules A from each cluster evenly in descending order of afᵢ. Further, b2 molecules B fragmented by the probability Prᵢ calculated using the equation (17) are selected (step 205). However, b2 is an integer of 1 to a1, preferably an integer of 1 to 1,000. The molecules B are selected only in the case of a crossover-reaction, and are not always selected from within the same cluster as the starting molecules A. The molecules B may be selected from different clusters.

$$\begin{matrix}[ Math . 17 ] & \\ {Pr}_{i} = \frac{{af}_{i}}{{\sum }_{i = 1}^{n} {e}^{{af}_{i}}} & ( 17 )\end{matrix}$$

The fragmented molecule is subdivided in units of one or more heavy atoms (step 206). The molecule is evolved by causing a crossover-reaction or mutation by adding an arbitrary atom, substituting an atom, or adding a fragment at an arbitrary position of the starting molecule. The newly generated molecule C is added to the phylogenetic tree of the starting molecule and classified into one of the f1 types of clusters (step 207). The cluster classification corresponds to the first generation.

The processes of steps 204 to 208 are repeated for all the newly generated molecules including the b1 molecules. At this time, for the molecule in which the newly generated molecules are added to its own phylogenetic tree, the afᵢ including the number of the added molecules is calculated as the confidence limit UCB1ᵢ using the equation (1) (step 204). At this time, in the equation (1), n is the sum of the number of molecules initially read and the number of newly generated molecules, nᵢ is the number of all molecules generated after the molecule to be calculated and added to the same phylogenetic tree, and the average value of xᵢ is the average value of the scores of all the molecules generated after the molecule to be calculated and added to the same phylogenetic tree. If there is only one molecule in the phylogenetic tree, the acquisition function value calculated using the equation (16) is used.

The molecule having the maximum acquisition function in each cluster of CL(1) to CL(f1) is selected as the next starting molecule. Specifically, the nᵢ at the time of the 5th generation of CL(2) in FIG. 5 is counted as 6 for the molecule A, 5 for C, 4 for D, 3 for E, and 1 for F and G, respectively.

The processes of steps 204 to 208 are repeated c times to generate a predetermined number of new molecules, and then a total of a1+b1×c molecules are classified into f2 clusters (step 210). Here, f2 is an integer and may be equal to or different from f1. c is an integer of 1 or more, and may preferably be in the range of 1 to 1,000,000,000.

The processes of steps 202 to 210 may be repeated a plurality of times to classify all the molecules into f3 clusters and end the operation. Here, f3 is an integer and may be equal to or different from f1 and f2. Further, a1 new molecules different from the a1 molecules used in step 201 may be selected from the database in which molecular structures are stored, and the above-mentioned processes may be repeated a plurality of times.

### <Specific Example of Molecular Structure Generation Method of Present Embodiment>

Specific examples of the process of generating a molecular structure having a maximum absorption at 500 to 600 nm by the molecular structure generation method of the present embodiment will be described below. The processing conditions in this specific example are as follows.

| | | |
|---|---|---|
| Molecular weight | 100 to 500 | Essential condition |
| Longest maximum | 0 to 1000 nm for first | Weight 0.4 |
| | desired region | |
| absorption wavelength | 500-600 nm for second | |
| | desired region | |
| Oscillator strength | 0.5 or more | Weight 0.4 |
| SA score | 1 to 4 | Weight 0.2 |

Molecular score = PR (λₘₐₓ) × 0.4 + PR (oscillator strength) × 0.4 + PR (SA score) × 0.2

The molecular structures read from the database and the evolved molecular structures are represented, for example, in SMILES. This SMILES structure was converted into a three-dimensional structure using RDkit in this specific example. Structural optimization was performed by the semi-empirical molecular orbital method PM6 method of Gaussian 16 using the three-dimensional coordinate data, and then 20 excitation energies were calculated by the ZINDO method. Further, each wavelength peak was covered with a Gaussian function to obtain a UV-VIS spectrum. The longest maximum absorption wavelength λₘₐₓ was estimated from this spectrum.

1,000 molecules were randomly selected as the initial structure from the database ZINC, the feature amounts of each molecule were extracted in 2,048 dimensions by Morgan Fingerprint, and the molecules were classified into 10 types of clusters CL(1) to CL(10) using the k-means++ method of scikit-learn. Structural optimization by Gaussian16/PM6 and excitation energy calculation by the ZINDO method were performed for 1,000 molecules to calculate λₘₐₓ, the scores of each molecule were calculated by UCB1, and 10 molecules were selected as the starting molecules and evolved. At this time, when the structures of 2,000 molecules were generated with a1=1000, b1=10, and c1=100, all the generated molecules were reclassified into 10 types of clusters CL(1) to CL(10). The above-described operation was performed again using the above-mentioned 2,000 molecules to generate 1,000 new molecules, and a total of 3,000 molecules were obtained. This operation was repeated 8 more times to generate a total of 11,000 molecular structures.

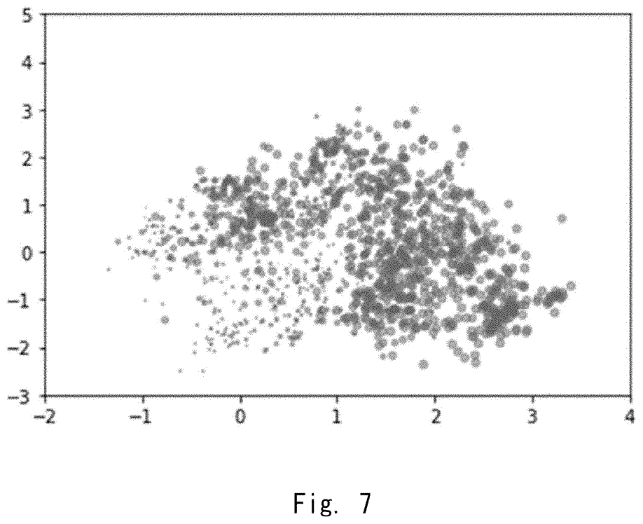

FIG. 7 shows the results of the principal component analysis for the molecular structure generated using the molecular structure generation method of the present embodiment. FIG. 7 shows a two-dimensional projection of 11,000 molecules generated by calculating the feature amount by Morgan Fingerprint and performing principal component analysis using scikit-learn.

According to the present embodiment, various molecular structures can be generated so as not to be localized around a specific molecular structure.

### Second Embodiment

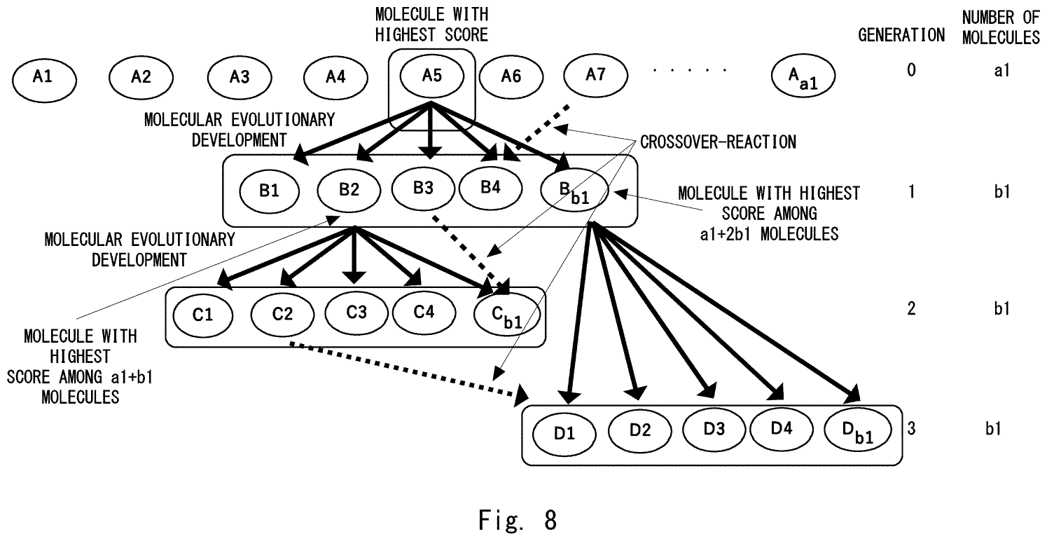

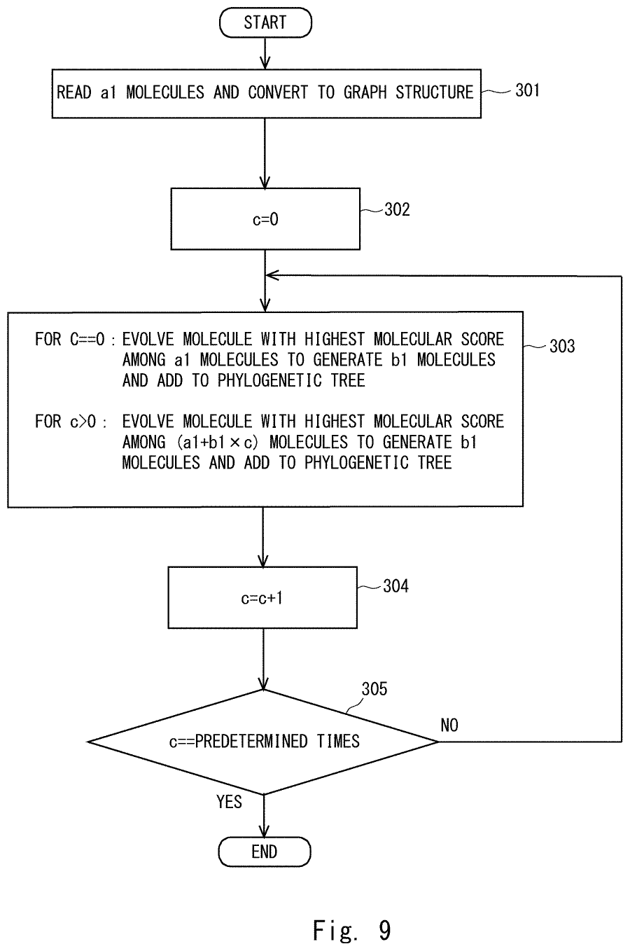

The molecular structure generation method of the present embodiment will be described with reference to FIGS. 8 and 9. FIG. 8 is a conceptual diagram showing a molecular structure generation method of the present embodiment. FIG. 9 is a flowchart of a process of generating a molecular structure in the present embodiment. In the present embodiment, the desired property value is PR.

As shown in FIG. 8, in the present embodiment, first, the score of each of any a1 molecules is calculated. Unlike the case of the first embodiment, the cluster classification is not performed. a1 may be, for example, 1,000, but is not limited to this. A molecule with the highest score is selected from a1 molecules and is evolved to generate b1 molecules. A molecule with the highest score is selected from a total of a1+b1 molecules and is evolved to generate b1 molecules. By repeating these processes a plurality of times, a predetermined number of molecules are generated. The molecule may be evolved by crossover-reacting or mutating with another molecule.

The flow of the process of generating the molecular structure in the present embodiment will be described with reference to FIG. 9. First, a1 molecular structures are read from a database in which molecular structures are stored, and converted to a graph structure that expresses a molecular structure using the atoms constituting the molecule as nodes and the bonds between atoms as edges and stored in a data frame (step 301). The a1 molecules correspond to the 0th generation.

The molecular score is calculated using the equation (16) for a1 molecules stored in a data frame. In addition, one molecule with the highest score is selected and molecular evolutionary development is performed. If a crossover-reaction is selected, the molecule to be fragmented is selected according to the probability calculated using equation (17). By these operations, b1 molecules are newly generated and added to the phylogenetic tree of the starting molecule (step 303). The b1 molecules correspond to the first generation.

The molecular score is calculated for a1+b1 molecules using the equation (1), and the molecule with the highest molecular score is used as the starting molecule and is evolved. If a crossover-reaction is selected, one molecule to be fragmented according to the probability calculated using the equation (17) is selected from molecules other than the starting molecule. By these operations, b1 molecules are newly generated and added to the phylogenetic tree of the starting molecule (step 303). The b1 molecules correspond to the second generation.

The process of step 303 is further repeated c-2 times, and when the addition of the phylogenetic tree is completed for a total of b1×c molecules (YES in step 305), the process is completed. Further, a1 new molecules different from the a1 molecules used in step 301 may be selected from the database in which molecular structures is stored, and the above-mentioned process may be repeated a plurality of times. Here, c is an integer of 1 or more, and may preferably be in the range of 1 to 1,000,000,000.

### <Specific Example of Molecular Structure Generation Method of Present Embodiment>

Specific examples of the process of generating a molecular structure having a maximum absorption at 500 to 600 nm by the molecular structure generation method of the present embodiment will be described below. The processing conditions in this specific example are the same as in the case of the first embodiment.

In the present embodiment, 1,000 molecules were randomly selected as the initial structure using ZINC, structural optimization by Gaussian16/PM6 and excitation energy calculation by the ZINDO method were performed to calculate λₘₐₓ, and the scores of each molecule were calculated by the equation (16). One molecule with the highest score was selected and evolved to generate ten new molecules. Next, UCB1ᵢ was calculated for 1,010 molecules using the equation (1) or (16), one molecule having the largest UCB1ᵢ was selected, and evolved to generate ten new molecules. This operation was further repeated 998 times to generate a total of 10,000 molecular structures.

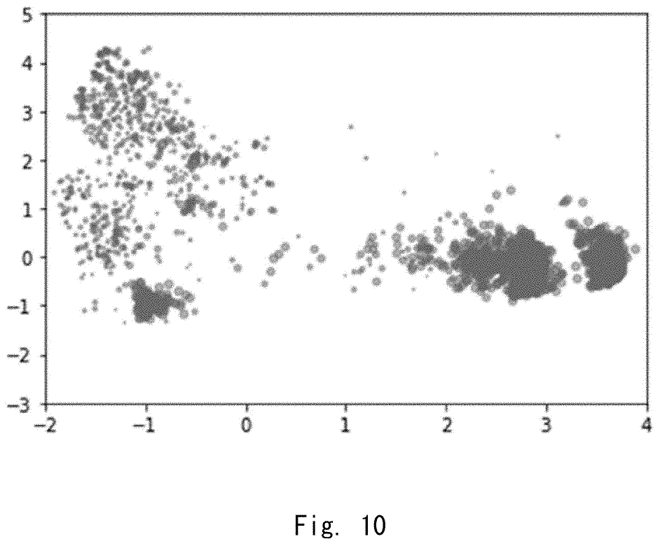

FIG. 10 shows the results of the principal component analysis for the molecular structure generated using the molecular structure generation method of the present embodiment. FIG. 10 shows a two-dimensional projection of 11,000 molecules generated by calculating feature amounts using Morgan Fingerprint and performing principal component analysis.

According to the present embodiment, various molecular structures can be generated so as not to be localized around a specific molecular structure. Further, unlike the case of the first embodiment, since the molecules are randomly selected and evolved without clustering, it is easier to secure the diversity of the generated molecules.

### Third Embodiment

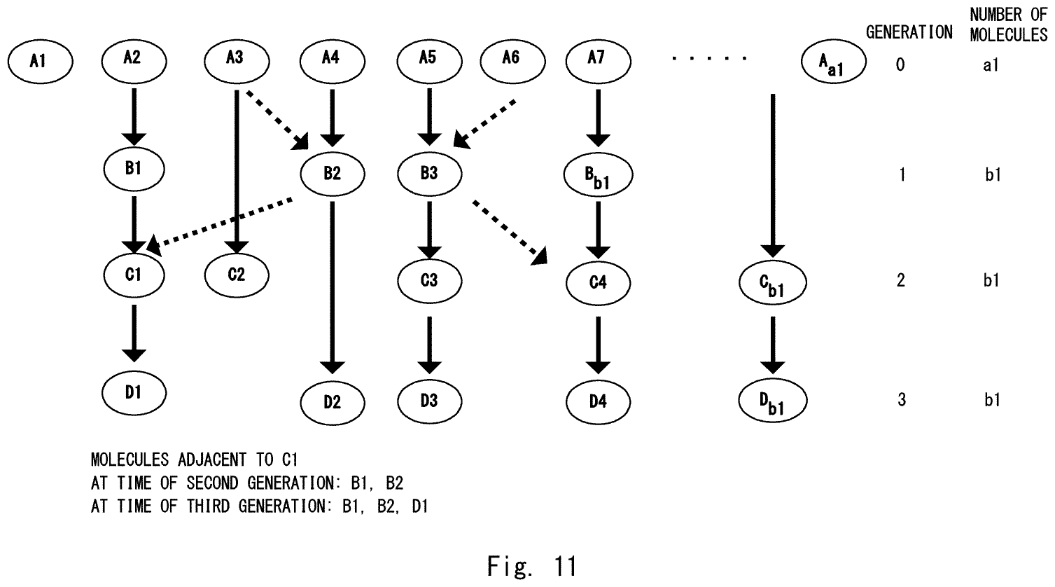

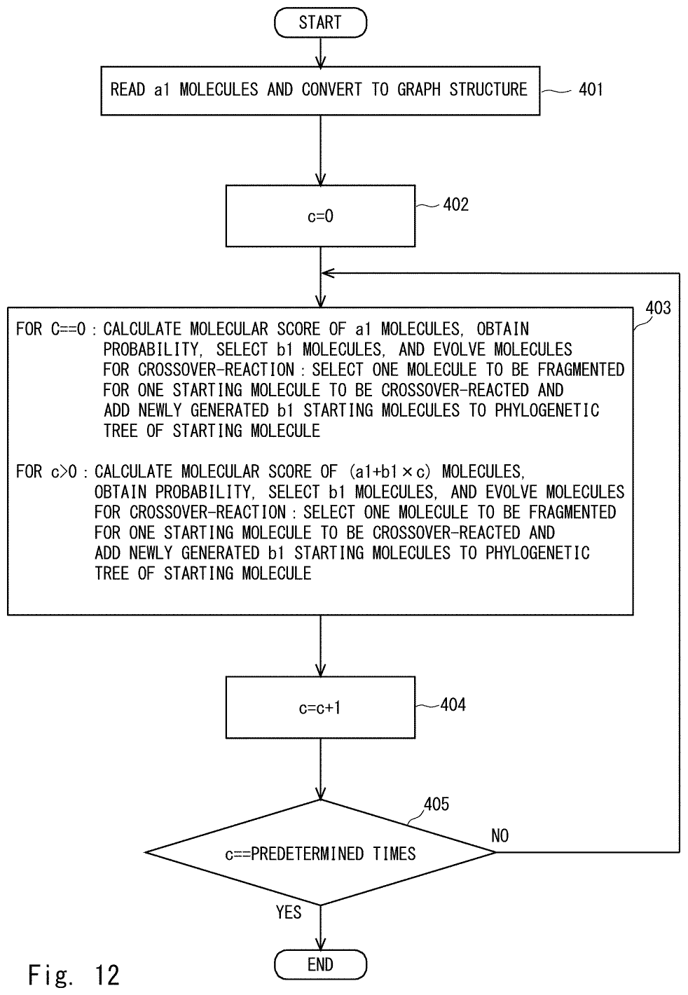

The molecular structure generation method of the present embodiment will be described with reference to FIGS. 11 and 12. FIG. 11 is a conceptual diagram showing a molecular structure generation method of the present embodiment. FIG. 12 is a flowchart of a process of generating a molecular structure in the present embodiment. In the present embodiment, the desired property value is PR.

As shown in FIG. 11, in the present embodiment, first, a molecular score is calculated for any a1 molecules, and a probability is obtained to select b1 molecules. Unlike the case of the first embodiment, the cluster classification is not performed. Further, unlike the case of the second embodiment, one molecule having the maximum molecular score is not selected. a1 may be, for example, 1,000, but is not limited to this. New b1 molecules are evolved. Further, b1 molecules are selected from a1+b1 molecules and further evolved to generate b1 molecules. By repeating these processes a plurality of times, a predetermined number of molecules are generated. The molecule may be evolved by crossover-reacting or mutating with another molecule.

The flow of the process of generating the molecular structure in the present embodiment will be described with reference to FIG. 12. First, a1 molecular structures are read from a database in which molecular structures are stored, and converted to a graph structure that expresses a molecular structure using the atoms constituting the molecule as nodes and the bonds between atoms as edges and stored in a data frame (step 401). The b1 molecules correspond to the 0th generation.

The score of each of the a1 molecules stored in the data frame is calculated from the first term on the right side of the equation (2), and a probability is obtained by the equation (4) to select b1 molecules. Evolutionary development is carried out for the b1 molecules. If a crossover-reaction is selected, one molecule to be fragmented is selected for one starting molecule according to the probability calculated using the equation (4). By these operations, b1 molecules are newly generated and added to the phylogenetic tree of the starting molecule (step 403). The b1 molecules correspond to the first generation.

The molecular score is calculated for a1+b1 molecules using the equation (2). When B1 is present in the phylogenetic tree as in A2 of FIG. 11, the adjacent molecule is counted as 1. If only one molecule is included in the phylogenetic tree, it is calculated by the first term only. A probability is obtained using the equation (4) to select b1 molecules from the a1+b1 molecules as the starting molecule, and the b1 molecules are evolved. If a crossover-reaction is selected, one molecule to be fragmented is selected for one starting molecule according to the probability calculated using equation (17). By these operations, b1 molecules are newly generated and added to the phylogenetic tree of the starting molecule (step 403). The b1 molecules correspond to the second generation.

The process of step 403 is repeated for a1+b1×2 molecules to generate new b1 molecules. At this time, the number of adjacent molecules of the molecule C1 in the second generation is two, B1 and B2 (step 403). The b1 molecules correspond to the third generation.

The process of step 404 is repeated for a1+b1×3 molecules, and further b1 molecules are newly generated (step 403). At this time, the number of adjacent molecules of the molecule C1 in the third generation is counted as 3, B1, B2, and D1.

The process of step 405 is repeated c-4 times, and when the addition of the phylogenetic tree is completed for a total of a1+b1×c molecules (YES in step 405), the process is completed. Further, a1 new molecules different from the a1 molecules used in step 401 may be selected from the database in which molecular structures is stored, and the above-mentioned process may be repeated a plurality of times. Here, c is an integer of 1 or more, and may preferably be in the range of 1 to 1,000,000,000.

### <Specific Example of Molecular Structure Generation Method of Present Embodiment>

Specific examples of the process of generating a molecular structure having a maximum absorption at 500 to 600 nm by the molecular structure generation method of the present embodiment will be described below. The processing conditions in this specific example are the same as in the case of the first embodiment.

1,000 molecules were randomly selected as the initial structure from ZINC, structural optimization by Gaussian16/PM6 and excitation energy calculation by the ZINDO method were performed to calculate λₘₐₓ, and the scores of each molecule were calculated by the first term on the right side of the equation (2). The probability of the scores was obtained using the equation (4) to select ten starting molecules which were evolved to generate new ten molecules. Next, the scores of for 1,010 molecules were calculated using the equation (2), and the probability was obtained using the equation (4) to select ten starting molecules, which were evolved. This operation was repeated 998 times to generate a total of 10,000 molecular structures.

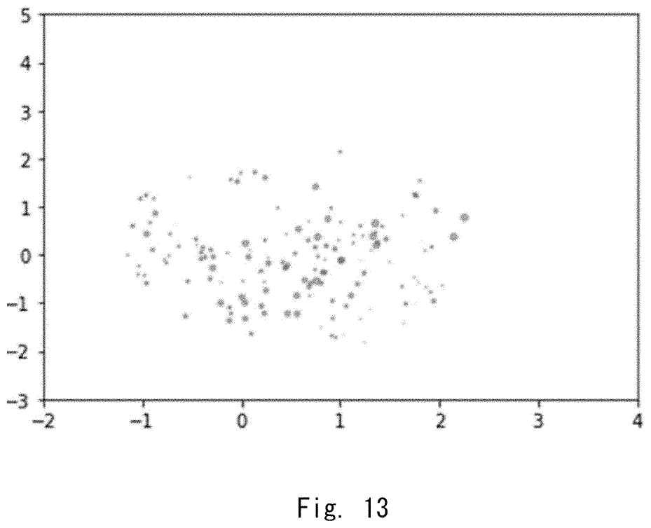

FIG. 13 shows the results of the principal component analysis for the molecular structure generated using the molecular structure generation method of the present embodiment. FIG. 13 shows a two-dimensional projection of 11,000 molecules generated by calculating the feature amounts using Morgan Fingerprint and performing principal component analysis.

According to the present embodiment, various molecular structures can be generated so as not to be localized around a specific molecular structure. Further, unlike the cases of the first and second embodiments, clustering is not performed and the molecule having the maximum molecular score is not evolved. Therefore, it is further easier to secure the diversity of the generated molecules as compared with the case of the second embodiment.

### <Comparison Between First to Third Embodiments and Conventional Example>

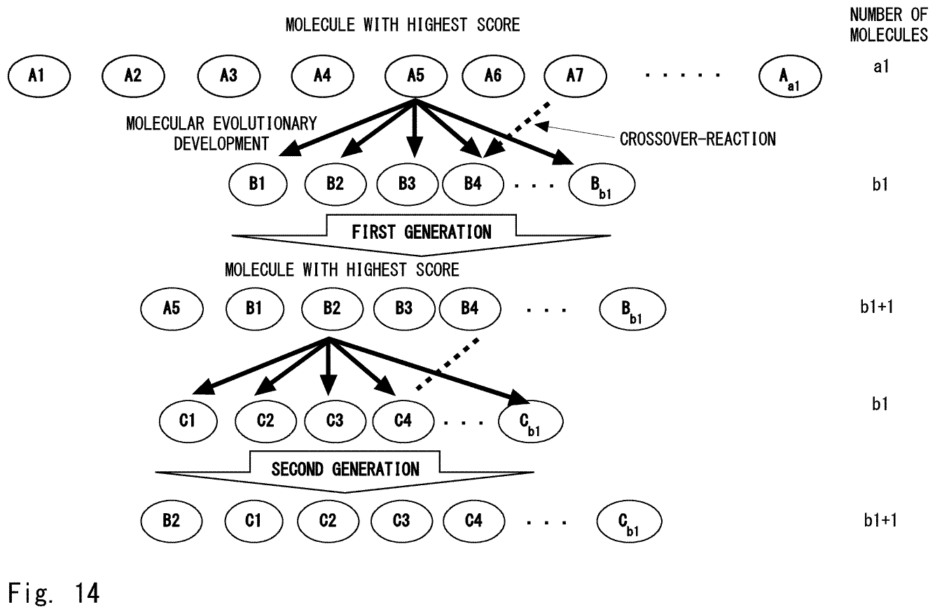

The molecular structure generated using the molecular structure generation method of the first to third embodiments will be compared with the molecular structure generated using the method according to the conventional example. FIG. 14 is a conceptual diagram showing a molecular structure generation method using a genetic algorithm method according to the conventional example.

As shown in FIG. 14, when a molecular structure is generated using a genetic algorithm method according to the conventional example, first, the score of each of any a1 molecules is calculated, and b1 molecules are generated from the molecule with the highest molecular score. Here, in the conventional example, unlike the first to third embodiments, a process of calculating the molecular score for only the newly generated b1 molecules and performing evolutionary development from the molecule with the highest molecular score is repeated. Therefore, the conventional example is different from the first to third embodiments in those molecules that are not selected as a molecule to be evolved are not the target for comparison of the molecular score, and are not the target for further evolutionary development.

### <Specific Example of Method of Generating Molecular Structure in Conventional Example>

A specific example of the process of generating a molecular structure having a maximum absorption at 500 to 600 nm according to the molecular structure generation method of the present embodiment will be described below. The conditions in this process are the same as in the case of the first embodiment.

1.000 molecules were randomly selected from ZINC as the initial structure, the scores of the molecules were calculated, and λₘₐₓ was calculated. For the molecular score, the value calculated by PR (λₘₐₓ)×0.4+PR (oscillator strength)×0.4+PR (SA score)×0.2 was used as it was. First, the molecule with the highest molecular score was selected from among 1,000 molecules as the starting molecule, and ten molecules were newly generated. At this time, the method of molecular evolutionary development is the same as that of the above-mentioned first to third embodiments. Next, the molecular scores were calculated for this starting molecule and ten newly generated molecules, one molecule having the highest score was newly selected, and ten molecules were generated by evolutionary development. This operation was repeated 998 times to generate a total of 10,000 molecular structures.

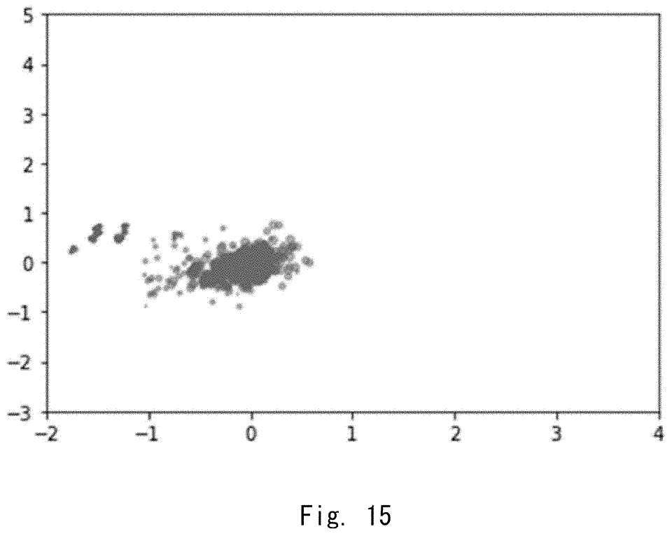

FIG. 15 shows the results of the principal component analysis for the molecular structure generated using the molecular structure generation method of the conventional example. FIG. 15 shows a two-dimensional projection of 11,000 molecules including 1,000 molecules as the initial structure and the generated 10,000 molecules, generated by calculating the feature amount by Morgan Fingerprint and performing principal component analysis.

The molecular distributions when the molecular structure generation methods of the first to third embodiments are used are widely distributed in the feature space as compared with the case shown in FIG. 15 using the conventional genetic algorithm method as shown in FIGS. 7, 10 and 13, respectively. Therefore, it can be said that various molecular structures can be generated.

### Other Embodiments

The molecular structure generation methods shown in the first to third embodiments can be widely used in inverse analysis for predicting a molecular structure having desired property values in various properties such as, for example, UV-VIS absorption spectrum, emission wavelength, dipole moment, polarizability, refractive index, dielectric constant, melting point, boiling point, lipophilicity, hydrophilicity, heat resistance, density, viscosity, elastic modulus, and dielectric constant contact.

### <Hardware Configuration Example>

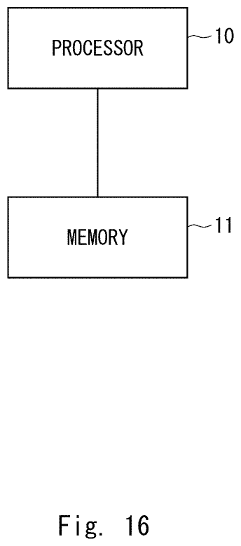

FIG. 16 is a block diagram showing a hardware configuration example for realizing the process related to the molecular structure generation method. The hardware configuration includes a processor 10 and a memory 11.

The processor 10 reads a computer program from the memory 11 and executes it to perform the process related to the molecular structure generation method described in the above-described embodiments. Here, the molecular structure generation program is a program that causes the information processing device 1 to execute: a selection process of selecting a starting molecule having the maximum confidence limit value from a plurality of initial molecules prepared in advance; an evolutionary development process of evolving each of the starting molecules; and a process of repeatedly executing the selection process and the evolutionary development process for all molecules including the initial molecules and the evolved starting molecules to generate a new molecular structure.

The molecular structure generation program is a program for causing the information processing device 1 to execute: a selection process of selecting a starting molecule having the maximum confidence limit value from a plurality of initial molecules prepared in advance; an evolutionary development process of evolving each of the starting molecules; and a process of repeatedly executing the selection process and the evolutionary development process for all molecules including the initial molecules and the evolved starting molecules to generate a new molecular structure.

The molecular structure generation program is a program that causes the information processing device 1 to execute: a selection process of calculating a feature amount of each of a plurality of initial molecules prepared in advance, and further selecting a starting molecule according to a probability value calculated based on the feature amount; an evolutionary development process of evolving each of the starting molecules; and a process of repeatedly executing the selection process and the evolutionary development process for all molecules including the initial molecules and the evolved starting molecules to generate a new molecular structure.

The processor 10 may be, for example, a microprocessor, an MPU (Micro Processing Unit), or a CPU (Central Processing Unit). The processor 200 may include a plurality of processors.

The memory 11 is composed of a combination of a volatile memory and a non-volatile memory. The memory 11 may include a storage located away from the processor 10. In this case, the processor 10 may access the memory 11 via an I/O interface (not shown).

In the example of FIG. 16, the memory 11 is used to store a group of software modules. The processor 10 reads these software modules from the memory 11 and executes them to perform the process related to the molecular structure generation method described in the above-described embodiments.

Each of the processors executes one or more programs including a group of commands for causing a computer to perform the algorithm described with reference to the drawings. This program can be stored and supplied to the computer using various types of non-transitory computer-readable media. Non-transient computer-readable media include various types of tangible storage media. Examples of non-transitory computer-readable media include magnetic recording media (for example, flexible disks, magnetic tapes, and hard disk drives), magneto-optical recording media (for example, magneto-optical disks), Compact Disc Read Only Memory (CD-ROM), CD-R, CD-R/W, semiconductor memory (for example, mask ROM, Programmable ROM (PROM), Erasable PROM (EPROM), flash ROM, and Random Access Memory (RAM)). The program may also be supplied to the computer by various types of transitory computer-readable media. Examples of transitory computer-readable media include electrical signal, optical signal, and electromagnetic waves. The transitory computer-readable media can supply the program to the computer via a wired communication path such as an electric wire and an optical fiber, or a wireless communication path.

The present disclosure is not limited to the above-described embodiments, and can be appropriately modified without departing from the spirit.

The first, second, third and other embodiments can be combined as desirable by one of ordinary skill in the art.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

1. A molecular structure generation method comprising:
a selection step of classifying a plurality of initial molecules prepared in advance into clusters based on a feature amount and selecting a starting molecule having a maximum confidence limit value from each of the classified clusters; and
an evolutionary development step of evolving each of the starting molecules,
wherein the selection step and the evolutionary development step are repeatedly executed for all molecules including the initial molecules and the evolved starting molecules to generate a new molecular structure.

2. A molecular structure generation method comprising:
a selection step of selecting a starting molecule having a maximum confidence limit value from a plurality of initial molecules prepared in advance; and
an evolutionary development step of evolving each of the starting molecules,
wherein the selection step and the evolutionary development step are repeatedly executed for all molecules including the initial molecules and the evolved starting molecules to generate a new molecular structure.

3. A molecular structure generation method comprising:
a selection step of calculating a feature amount of each of a plurality of initial molecules prepared in advance and further selecting a starting molecule according to a probability value calculated based on the feature amount; and
an evolutionary development step of evolving each of the starting molecules,
wherein the selection step and the evolutionary development step are repeatedly executed for all molecules including the initial molecules and the evolved starting molecules to generate a new molecular structure.

4. The molecular structure generation method according to claim 1, wherein
the molecular structure is represented using a graph notation in which atoms constituting a molecule are expressed as nodes and bonds between the atoms are expressed as edges.

5. The molecular structure generation method according to claim 2, wherein
the molecular structure is represented using a graph notation in which atoms constituting a molecule are expressed as nodes and bonds between the atoms are expressed as edges.

6. The molecular structure generation method according to claim 3, wherein
the molecular structure is represented using a graph notation in which atoms constituting a molecule are expressed as nodes and bonds between the atoms are expressed as edges.

7. The molecular structure generation method according to claim 1, wherein
the evolutionary development is caused by crossover-reaction or mutation.

8. A non-transitory computer-readable medium storing a program for causing an information processing device to execute processes, the processes comprising:
a selection process of classifying a plurality of initial molecules prepared in advance into clusters based on a feature amount and selecting a starting molecule having a maximum confidence limit value from each of the classified clusters; and
an evolutionary development process of evolving each of the starting molecules,
wherein the selection process and the evolutionary development process are repeatedly executed for all molecules including the initial molecules and the evolved starting molecules to generate a new molecular structure.

9. A non-transitory computer-readable medium storing a program for causing an information processing device to execute processes, the processes comprising:
a selection process of selecting a starting molecule having a maximum confidence limit value from a plurality of initial molecules prepared in advance; and
an evolutionary development process of evolving each of the starting molecules,
wherein the selection process and the evolutionary development process are repeatedly executed for all molecules including the initial molecules and the evolved starting molecules to generate a new molecular structure.

10. A non-transitory computer-readable medium storing a program for causing an information processing device to execute processes, the processes comprising:
a selection process of calculating a feature amount of each of a plurality of initial molecules prepared in advance and further selecting a starting molecule according to a probability value calculated based on the feature amount; and
an evolutionary development process of evolving each of the starting molecules,
wherein the selection process and the evolutionary development process are repeatedly executed for all molecules including the initial molecules and the evolved starting molecules to generate a new molecular structure.

11. The non-transitory computer-readable medium storing a program according to claim 8, wherein
the molecular structure is represented using a graph notation in which atoms constituting a molecule are expressed as nodes and bonds between the atoms are expressed as edges.

12. The non-transitory computer-readable medium storing a program according to claim 9, wherein
the molecular structure is represented using a graph notation in which atoms constituting a molecule are expressed as nodes and bonds between the atoms are expressed as edges.

13. The non-transitory computer-readable medium storing a program according to claim 10, wherein
the molecular structure is represented using a graph notation in which atoms constituting a molecule are expressed as nodes and bonds between the atoms are expressed as edges.

14. The non-transitory computer-readable medium storing a program according to claim 8, wherein
the evolutionary development is caused by crossover-reaction or mutation.

15. The molecular structure generation method according to claim 2, wherein
the evolutionary development is caused by crossover-reaction or mutation.

16. The molecular structure generation method according to claim 3, wherein
the evolutionary development is caused by crossover-reaction or mutation.

17. The molecular structure generation method according to claim 4, wherein
the evolutionary development is caused by crossover-reaction or mutation.

18. The molecular structure generation method according to claim 5, wherein
the evolutionary development is caused by crossover-reaction or mutation.

19. The molecular structure generation method according to claim 6, wherein
the evolutionary development is caused by crossover-reaction or mutation.

20. The non-transitory computer-readable medium storing a program according to claim 9, wherein
the evolutionary development is caused by crossover-reaction or mutation.

21. The non-transitory computer-readable medium storing a program according to claim 10, wherein
the evolutionary development is caused by crossover-reaction or mutation.

22. The non-transitory computer-readable medium storing a program according to claim 11, wherein
the evolutionary development is caused by crossover-reaction or mutation.

23. The non-transitory computer-readable medium storing a program according to claim 12, wherein
the evolutionary development is caused by crossover-reaction or mutation.

24. The non-transitory computer-readable medium storing a program according to claim 13, wherein
the evolutionary development is caused by crossover-reaction or mutation.
